(19) United States
(12) Plant Patent Application Publication
(10) Pub. No.: US 2018/0255673 P1
(43) Pub. Date: Sep. 6, 2018

(54) STRAWBERRY PLANT NAMED 'REDSTART'

(50) ;

(71) Applicant: Board of Trustees of Michigan State University, East Lansing, MI (US)

(72) Inventor: James Hancock, Scarborough, ME (US)

(21) Appl. No.: 15/530,811

(22) Filed: Mar. 3, 2017

(51) Int. Cl. A01H 6/74

(52) U.S. Cl. PLT/209

(57) ABSTRACT

A new and distinct Fragaria x ananassa plant named ‘Redstart’, particularly characterized as a weak day-neutral long fruiting variety.

Botanical name qf the genus and species of the plant claimed: The strawberry cultivar of this invention is botanically identified as Fragaria x ananassa.

Variety denomination: The variety denomination is ‘Redstart’.

### BACKGROUND OF THE INVENTION

The present invention relates to a new and distinct variety of a strawberry plant, botanically known as Fragaria x ananassa of the Rosaceae family, and hereinafter referred to by the variety denomination ‘Redstart’.

The new Fragaria x ananassa variety is a product of a planned breeding program conducted by the inventors. The objective of the breeding program was to develop a new Fragaria x ananassa variety intended for the Midwestern and Northeastern USA, Ontario, and Quebec and the Pacific Northwest. It is a weak day-neutral, and has a long fruiting season.

The new variety originated from a cross between ‘Honeoye’ and ‘Chandler’ (U.S. Plant Pat. No. 5,262) made in a greenhouse at Michigan State University East Lansing, Mich. in March 2006. Redstart was first selected in the summer of 2007 from a family of 76 planted in an open filed at the Southwest Michigan Research and Extension Center (SWMREC), Benton Harbor, Mich. It was re-evaluated in the same field the following spring and was then designated as MSU 67.

Asexual propagation of the new Fragaria x ananassa variety by runners was first performed at Michigan State University East Lansing Mich. in the fall of 2008, where runners from the original mother plant were dug up and transferred to a greenhouse at Michigan State. The potted plants were allowed to runner in the summer of 2011 and 2013 and the resulting daughter plants were rooted in sterilized soil. These plants were encouraged to go dormant each winter by leaving the greenhouse unheated.

Horticultural examination has demonstrated that the combination of characteristics as herein disclosed for the new variety was firmly fixed and retained through successive generations of asexual propagation. The new variety propagates true-to-type.

### BRIEF DESCRIPTION OF THE INVENTION

The following traits have been repeatedly observed and are determined to be characteristics of ‘Redstart’, which in combination, distinguish this strawberry plant as a new, unique and distinct variety. ‘Redstart’ is a vigorous weak day-neutral variety, with high yields and excellent fruit quality.

In comparison to the parental varieties, ‘Honeoye’ and ‘Chandler’ (U.S. Plant Pat. No. 5,262), ‘Redstart’ differs primarily in the traits listed in Table 1.

TABLE 1
Comparison with parent varieties

| | Female | Male | |
|---|---|---|---|
| | Parent | Parent | |
| Trait | ′Honeoye′ | ′Chandler′ | ′Redstart′ |
| Fruiting | Short-day | Short-day | Day-neutral |
| season | | | |
| Plant vigor | High | Medium | High |
| Fruit shape | Conic | Long conic | Conic |
| Fruit color | Dark red | Medium red | Medium red |

Of the many commercial varieties known to the present inventors, the most similar in comparison to the new Fragaria x ananassa ‘Redstart’ is Fragaria x ananassa ‘Albion’, (U.S. Plant Pat. No. 16,228) or ‘Seascape’ (U.S. Plant Pat. No. 7,614). ‘Redstart’ proved to be more vigorous, had higher yields and better fruit color than ‘Albion’, although it was smaller fruited and less firm. Their fruit flavor was comparable. ‘Redstart’ had higher vigor and yields than ‘Seascape’, better tasting fruit and comparable firmness. Its fruit were paler than ‘Seascape’. Description of field trials is below.

### Trials

Ten plants of MSU 67 were planted in 2012 and 2014 with other MSU selections in an open field at SWMREC and at Sakuma Brothers Farms, Burlington, Wash. under high tunnels. Plants were maintained on a black plastic mulch in raised beds at Sakuma's, while at SWMREC they were grown on flat beds without mulch. All flowers were removed from the first flush of blooms in the planting year. Adjacent to these trials were other plantings of the day-neutral cultivars ‘Albion’ (U.S. Plant Pat. No. 16,228) and ‘Seascape’ (U.S. Plant Pat. No. 7,614). The plants set in 2012 were evaluated in years one and two for weeks of fruiting, vigor, fruit size, productivity and fruit quality. The plants set in 2014 were evaluated for the same parameters in just that year.

### Results

### Michigan Field Trials (2012-2013)

In the open fields in Michigan in 2012 and 2013, ‘Redstart’ was acceptable to superior for all the rated characteristics (Table 2). It fruited for 6 weeks in 2012 and 5 weeks in 2013. In the first fruiting season (Table 2), ‘Redstart’ was more vigorous than ‘Albion’ and had better fruit color. It had similar yields, fruit appearance and flavor, but its fruit were smaller and softer. ‘Redstart’ out-yielded ‘Seascape’ and was better flavored, had comparable fruit size and appearance, but had a little less internal fruit color and was slightly softer.

In the second fruiting season, ‘Redstart’ had much higher vigor and yields than ‘Albion’, and had comparable fruit appearance, color and flavor. Its fruit were not as firm and were smaller. ‘Redstart’ was more vigorous than ‘Seascape’ and had larger, more attractive fruit with better flavor. It had lower yields than ‘Seascape’ and was less colored.

TABLE 2
Strawberry trials in the open field at the Southwest
Michigan Research and Extension Center
(Benton Harbor, MI) in 2012 and 2013.

| | | Cultivars | | |
|---|---|---|---|---|
| Year | Characteristic | ′Albion′ | ′Redstart′ | ′Seascape′ |
| 2012 | Plant vigor | 7^{a} | 8 | 8 |
| | Total Yield | 9 | 9 | 7 |
| | Fruit size | 9 | 7 | 7 |
| | Fruit appearance | 8 | 8 | 8.5 |
| | External color | 6 | 7 | 8 |
| | Internal color | 7 | 8 | 8 |
| | Firmness | 9 | 7 | 8 |
| | Flavor | 8 | 8 | 6 |
| 2013 | Plant vigor | 4 | 8 | 6 |
| | Total Yield | 3 | 6 | 7 |
| | Fruit size | 7 | 6 | 3 |
| | Fruit appearance | 8 | 8 | 7 |
| | External color | 7 | 8 | 9 |
| | Internal color | 7 | 8 | 8 |
| | Firmness | 10 | 8 | 8 |
| | Flavor | 7 | 7 | 6 |

^{a}1 = poor; 7 = commercially acceptable, 10 = excellent

### Washington Tunnel Trials (2012-2Q13)

In the tunnel trials in Washington in 2012 and 2013 (Table 3), ‘Redstart’ was acceptable to superior for all the rated characteristics, except fruit size in 2013. It fruited for 11 weeks in 2012 and 13 weeks in 2013.

In the first fruiting season under the hoops in Mt. Vernon, ‘Redstart’ had higher vigor and yields than ‘Albion’, but was less firm and not quite as flavorful. They had comparable color and appearance. ‘Redstart’ was more vigorous than ‘Seascape’, had larger fruit and was more flavorful. The fruit of ‘Seascape’ had less color than ‘Redstart’ and was slightly less firm.

In the second harvest season, ‘Redstart’ was much more vigorous than ‘Albion’ and had better color. ‘Albion’ was larger fruited and more firm, and they were comparable in yield. ‘Redstart’ had more vigor than ‘Seascape’ and had slightly lower yields and fruit weight. They were comparable in fruit color, firmness and flavor.

TABLE 3
Strawberry trials in hoop houses at Sakuma Brothers
Farms (Mt. Vernon, WA) in 2012 and 2013.

| | | Cultivars | | |
|---|---|---|---|---|
| Year | Characteristic | ′Albion′ | ′Redstart′ | ′Seascape′ |
| 2012 | Plant vigor | 7^{a} | 8 | 7 |
| | Total Yield | 8 | 9 | 9 |
| | Fruit size | 9 | 8 | 7 |
| | Fruit appearance | 8 | 8 | 8 |
| | External color | 8 | 8 | 9 |
| | Internal color | 6 | 6 | 8 |
| | Firmness | 10 | 8 | 9 |
| | Flavor | 8 | 7 | 6 |
| 2013 | Plant vigor | 8 | 10 | 8 |
| | Total Yield | 8 | 8 | 9 |
| | Fruit size | 10 | 6 | 7 |
| | Fruit appearance | 8 | 7 | 8 |
| | External color | 8 | 8 | 8.5 |
| | Internal color | 6 | 8 | 8 |
| | Firmness | 10 | 8 | 8 |
| | Flavor | 9 | 8 | 8 |

^{a}1 = poor, 7 = commercially acceptable, 10 = excellent

### Michigan Field Trials and Washington Tunnel Trials in 2014

In both Michigan and Washington in 2014, all characteristics of ‘Redstart’ were rated acceptable to superior. In Michigan, ‘Redstart’ fruit were smaller than ‘Albion’ and less firm, but they had better color and their plants had higher vigor and yields; ‘Redstart’ and ‘Albion’ were comparable in flavor and fruit appearance. In Washington, ‘Redstart’ fruit were less firm and smaller than ‘Albion’, but they had better appearance and internal color, and the plants had higher yields.

TABLE 4
Strawberry trials in the open field at
Salcuma Brothers Farms (Mt. Vernon, WA)
and the Southwest Michigan Research and
Extension Center (Benton Harbor, MI) in 2014.

| | | Cultivar | |
|---|---|---|---|
| Location | Characteristic | ′Albion′ | ′Redstart′ |
| MI | Plant vigor | 7^{a} | 8′ |
| | Total Yield | 7 | 8 |
| | Fruit size | 8 | 7 |
| | Fruit appearance | 8 | 8 |
| | External color | 6 | 8 |
| | Internal color | 7 | 7 |
| | Firmness | 9 | 7 |
| | Flavor | 8 | 8 |
| WA | Plant vigor | 8 | 7 |
| | Total Yield | 7 | 8 |
| | Fruit size | 8 | 7 |
| | Fruit appearance | 7 | 8 |
| | External color | 9 | 7 |
| | Internal color | 7 | 8 |
| | Firmness | 9 | 7 |
| | Flavor | 8 | 8 |

^{a}1 = poor; 7 = commercially acceptable, 10 = excellent

### BRIEF DESCRIPTION OF THE PHOTOGRAPHS

The accompanying photographs illustrate the overall appearance of the new Fragaria x ananassa variety ‘Redstart’ showing the colors as true as is reasonably possible with colored reproductions of this type. Colors in the photographs may differ slightly from the color values cited in the detailed morphological description, which accurately describe the color of ‘Redstart’.

FIG. 1 shows a close up of a typical fruit bearing plant of ‘Redstart’ with flowers.

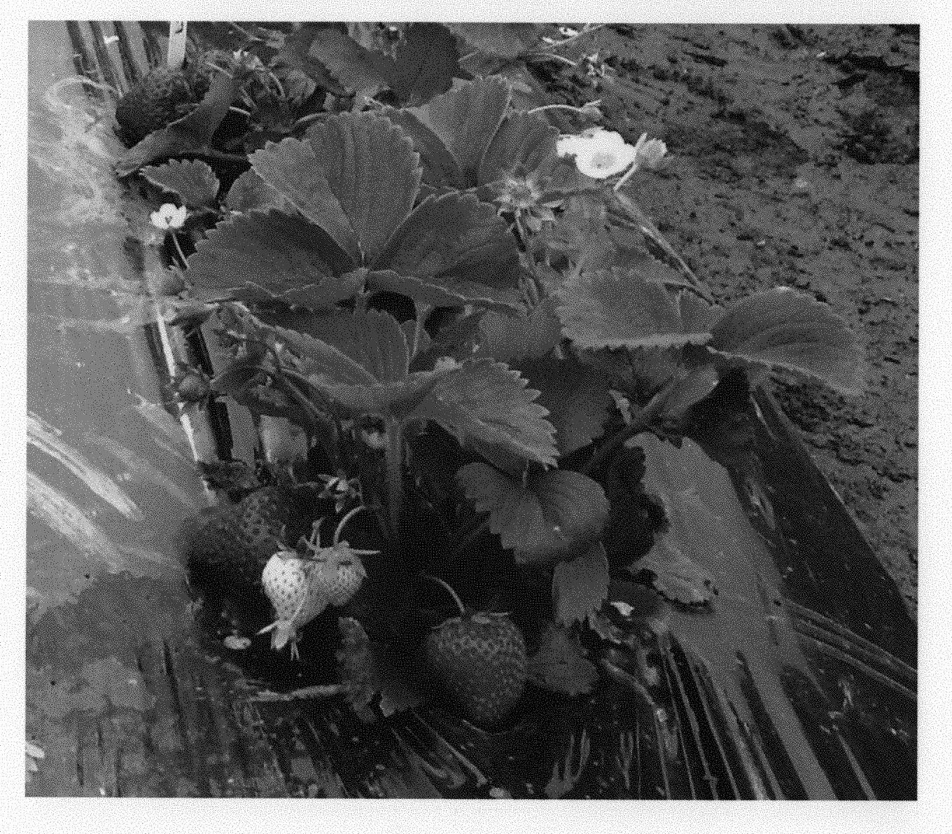

FIG. 2 shows a typical row of ‘Redstart’ planted in black plastic mulch.

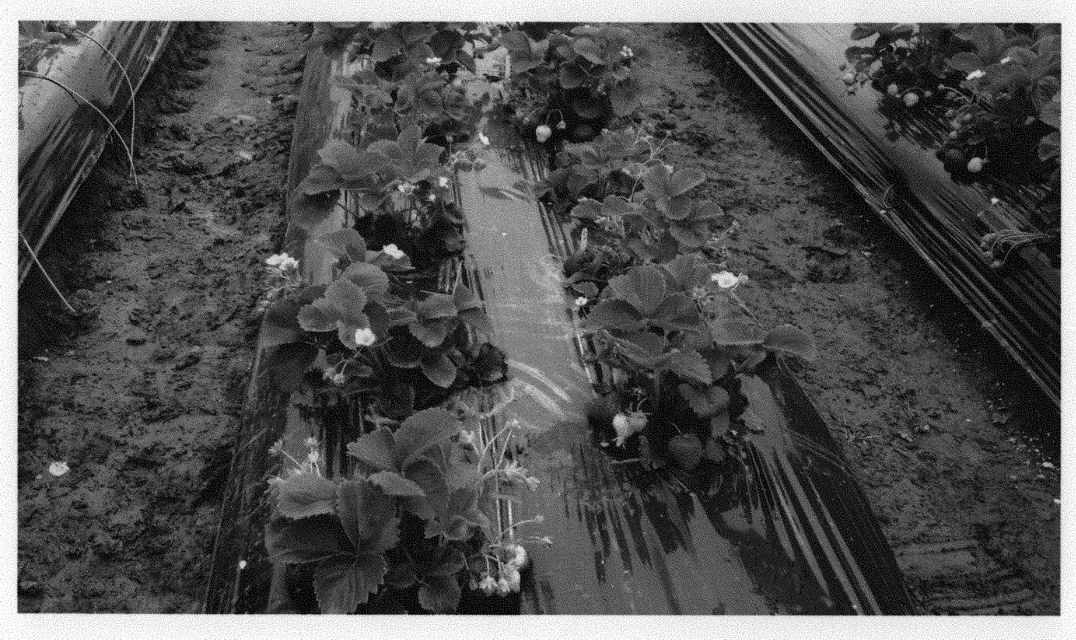

### DETAILED BOTANICAL DESCRIPTION

The new Fragaria x ananassa ‘Redstart’ has not been observed under all possible environmental conditions. The phenotype of the new variety may vary with variations in environment such as temperature, light intensity, day length or soil without any change in the genotype of the strawberry plant.

The aforementioned photographs, together with the following observations, measurements and values describe plants of ‘Redstart’ as grown in the field in Benton Harbor, Mich., under conditions which closely approximate those generally used in commercial practice. The described plants were propagated from stolons and planted at a distance of 25 cm in sandy red foam soil at an elevation of about 30 meters above sea level, with drip irrigation and fertilizers as generally used in commercial practice. Average annual precipitation is about 550 mm, with an average 350 mm of precipitation in winter (December to February). Mean diurnal minimum temperature in January is −8° C., and mean diurnal maximum temperature in July is 24° C.

Mother plants were planted in the beginning of May, in the field nursery at 2.5 m×2.5 m distance. Overhead irrigation with addition of fertilizers was used. The average day/night temperatures during the establishment of the daughter plants, between June to August, are 32° C./18° C., respectively. Runners appeared from June, which produce young daughter plants up till mid-September

Unless otherwise stated, the detailed morphological description includes observations, measurements and values taken from May to June, 2014 and based on ‘Redstart’ plants grown in a greenhouse at Michigan State University. Quantified measurements are expressed as an average or a range of measurements taken from a number of plants of ‘Redstart’. The measurements of any individual plant or any group of plants, of the new variety may vary from the stated average or range.

Color references are made to The Royal Horticultural Society Colour Chart (R.H.S.), (1986 edition), except where general colors of ordinary significance are used. All of the plants of ‘Redstart’, insofar as they have been observed, have been consistent in all the characteristics described below.

TABLE OF CHARACTERISTICS

```
Classification:
  Botanical.—Fragaria × ananassa
Parentage:
  Female or seed parent.—′Honeoye′ (Unpatented)
  Male or pollen parent.—′Chandler′ (U.S. Plant Pat. No. 5,262)
Propagation:
  By runners in a greenhouse at Michigan State University
Foliar characteristics in mid—summer:
  Plant height (mm).—18.4 (14-23)
  Plant spread.—29.2 (25-31)
  Color.—141B
  Mid-tier leaflet length (mm).—73.2 (60-81)
  Shape (length/width).—64.6 (62-73)
  Basal angle of terminal leaflet.—10.2 (8-12)
  Serrations of terminal leaflet.—24.6 (23-26)
  Serrations per leaf—73.8 (70-84)
  Petiolule length (mm).—9.6 (6-11)
  Petiole length.—129.2 (108-138)
  Petiole diameter.—2.2 (2.0-2.5)
  Petiole color.—145A
  Venation.—Pinnate
  Leaf pubescence.—None to sparse
  Petiole pubescence.—Sparse to moderate
  Petiole pubescence direction.—Perpendicular
Flowering and fruiting characteristics:
  Flowers per truss.—6.1 (5-7)
  Flower position.—Most exposed
  Petal width (mm).—10.5 (10-11.5)
  Calyx diameter.—23.2 (21-27)
  Corolla diameter.—28.2 (22-33)
  Sepal length.—11.3 (10.5-14.5)
  Sepal width.—5.6 (4.5-7.0)
  Sepal color.—147A
  Pedicle length (mm).—18.9 (13-24.5)
  Pedicle diameter (mm).—1.3 (1.1-2.0)
  Pedicle color.—146D
  Fruit width.—33.6 (31.5-35.50)
  Fruit length.—37.7 (35-40)
  Length/width.—1.1 (1.0-1.3)
  Fruit shape.—Conic to long conic
  Size (g/frt).—13.2 (10.2-14.8)
  Calyx diameter.—28.8 (24-35)
  Calyx postion.—Even
  External color.—45A
  Internal
  color.—45A
  Depth of color.—50%
  Fruit per truss.—6.1 (5-7)
  Achene color.—163B (pale tan)
  Achene placement.—Level
Fruit quality measurements:
  SS.—8.2
  TA.—9.2 (9.0-9.4)
```

What is claimed is:

1. A new and distinct variety of Fragaria x ananassa plant named ‘Redstart’, as illustrated and described herein.
